(19) United States
(12) Patent Application Publication
(10) Pub. No.: US 2026/0117164 A1
(43) Pub. Date: Apr. 30, 2026

(54) ANGLED RIDGE MICROFLUIDIC FILTER FOR CONTINUOUS BUFFER EXCHANGE OF SUSPENDED CELLS

(71) Applicant: Georgia Tech Research Corporation, Atlanta, GA (US)

(72) Inventors: Todd Sulchek, Atlanta, GA (US); Steven Swingle, Atlanta, GA (US); Alexander Alexeev, Atlanta, GA (US)

(21) Appl. No.: 19/368,333

(22) Filed: Oct. 24, 2025

Related U.S. Application Data
(60) Provisional application No. 63/711,533, filed on Oct. 24, 2024.

(51) Int. Cl. C12M 1/00; C12M 3/06

(52) U.S. Cl. CPC C12M 47/04; C12M 23/16; C12M 29/04; C12M 29/14

(57) ABSTRACT

The present disclosure provides a microfluidic device for buffer exchange of suspended particles comprising a channel having a first inlet for a first fluid containing suspended particles and a second inlet for a second fluid. The device includes an angled ridge positioned within the channel at 5 degrees or less relative to a channel wall parallel to flow direction, extending partially across channel width and configured to deflect particles from the first fluid into the second fluid while allowing fluids to pass through a ridge gap. A gutter region adjacent to the angled ridge distal end allows particles to pass through. An outlet collects particles transferred into the second fluid.

### CROSS-REFERENCE TO RELATED APPLICATIONS

This application claims the benefit of U.S. Provisional Application Ser. No. 63/711,533, filed on 24 Oct. 2024, which is incorporated herein by reference in its entirety as if fully set forth below.

### GOVERNMENT LICENSE RIGHTS

This invention was made with government support under 2134701 awarded by National Science Foundation. The government has certain rights in this invention.

### FIELD OF INVENTION

The present disclosure relates to microfluidic devices for particle processing, and more particularly to an angled ridge microfluidic filter for continuous buffer exchange of suspended cells and particles.

### BACKGROUND

Buffer exchange is a fundamental unit operation in cell processing and biotechnology applications. Traditional methods for exchanging the fluid medium surrounding suspended cells typically rely on centrifugation-based approaches, where cells are pelleted by centrifugal force, the supernatant is removed, and the cells are resuspended in fresh medium. While widely used, these conventional techniques present several limitations including manual labor requirements, discontinuous processing that interrupts workflows, potential cell loss during handling, and the need for bulky equipment that occupies substantial laboratory space.

The growing demand for automated and continuous cell processing has driven interest in microfluidic approaches for buffer exchange. Microfluidic devices offer advantages including reduced sample volumes, precise fluid control, and the potential for integration into automated systems. Various microfluidic strategies have been developed for cell processing, including approaches based on hydrodynamic focusing, deterministic lateral displacement, inertial manipulation, and filtration mechanisms.

Existing microfluidic buffer exchange methods often face challenges related to processing throughput, device complexity, and the balance between separation efficiency and cell viability. Some approaches require multiple fluid inlets or complex channel geometries that can complicate device fabrication and operation. Others may be limited in their ability to process cells across a range of sizes or may require specific flow rate conditions that restrict operational flexibility.

The field continues to seek improved microfluidic approaches that can provide efficient buffer exchange while maintaining high cell recovery rates, preserving cell viability, and offering operational simplicity. Advances in this area could benefit applications ranging from routine cell culture maintenance to specialized procedures such as cell preparation for electroporation, removal of cryopreservants from thawed cells, and transfer of cells between different media formulations for various downstream processes.

### SUMMARY

This summary is provided to introduce a selection of concepts in a simplified form that are further described below in the detailed description. This summary is not intended to identify key features or essential features of the claimed subject matter, nor is it intended to be used as an aid in determining the scope of the claimed subject matter.

According to an aspect of the present disclosure, a microfluidic device for buffer exchange of suspended particles is provided. The device comprises a channel having a first inlet configured to receive a first fluid containing suspended particles and a second inlet configured to receive a second fluid. The device comprises an angled ridge positioned within the channel at an angle of 5 degrees or less relative to a channel wall parallel to a direction of flow of the first and second fluids. The angled ridge extends partially across a width of the channel and is configured to deflect particles from the first fluid into the second fluid while allowing the first and second fluids to pass by the angled ridge through a ridge gap. The device comprises a gutter region adjacent to a distal end of the angled ridge, the gutter region sized to allow the particles to pass through the gutter region. The device comprises at least one outlet configured to collect particles transferred into the second fluid.

According to other aspects of the present disclosure, the device may include one or more of the following features. The angled ridge may be positioned at an angle of 2 degrees or less relative to the channel wall. The suspended particles may comprise cells. The gutter region may have a width that is at least three times larger than an average diameter of the cells. The channel may have a width in a range of 100 microns to 1 millimeter. The ridge gap may have a width smaller than a smallest diameter of the suspended particles to prevent particles from passing underneath the angled ridge. The ridge gap may be approximately one-third of an average diameter of the suspended particles.

According to another aspect of the present disclosure, a method for continuous buffer exchange of suspended particles is provided. The method comprises introducing a first fluid containing suspended particles into a first inlet of a microfluidic channel. The method comprises introducing a second fluid into a second inlet of the microfluidic channel, wherein the first fluid and second fluid flow in parallel within the channel under laminar flow conditions. The method comprises deflecting the suspended particles from the first fluid into the second fluid using an angled ridge positioned within the channel at an angle of 5 degrees or less relative to a channel wall parallel to a direction of flow of the first and second fluids, wherein the angled ridge extends partially across a width of the channel. The method comprises allowing the first fluid to pass by the angled ridge through a ridge gap while the particles are deflected along the angled ridge. The method comprises collecting the particles suspended in the second fluid from an outlet of the channel.

According to other aspects of the present disclosure, the method may include one or more of the following features. The angled ridge may be positioned at an angle of 2 degrees or less relative to the channel wall. The suspended particles may comprise cells. The ridge gap may be smaller than a smallest diameter of the cells to prevent cells from passing through the ridge gap. The ridge gap may have a width that is approximately one-third of an average diameter of the cells. The first fluid and second fluid may be maintained under laminar flow conditions. The method may further comprise a step of concentrating the particles by collecting only a portion of the second fluid containing the deflected particles while discarding remaining portions of both the first fluid and second fluid.

According to another aspect of the present disclosure, a microfluidic system for particle processing is provided. The system comprises a microfluidic channel having multiple inlets and multiple outlets. The system comprises an angled constriction element positioned within the channel at an angle relative to fluid flow direction, the angled constriction element having a gap dimension smaller than a diameter of particles to be processed. The system comprises a fluid delivery system configured to introduce two different fluids into the channel through separate inlets. The system comprises a collection system configured to collect processed particles from a designated outlet, wherein the angled constriction element is configured to guide particles from a first fluid stream into a second fluid stream while maintaining laminar flow separation between the fluid streams in the microfluidic channel.

According to other aspects of the present disclosure, the system may include one or more of the following features. The angled constriction element may be positioned at an angle of 5 degrees or less relative to a channel wall parallel to the fluid flow direction of the first and second fluid streams. The angled constriction element may be positioned at an angle of 2 degrees or less relative to the channel wall. The particles may comprise cells. The gap dimension may be approximately one-third of an average diameter of the cells. The fluid delivery system may comprise syringe pumps configured to maintain laminar flow conditions of the first and second fluid streams.

These and other aspects of the present disclosure are described in the Detailed Description below and the accompanying drawings. Other aspects and features of embodiments will become apparent to those of ordinary skill in the art upon reviewing the following description of specific, exemplary embodiments in concert with the drawings. While features of the present disclosure may be discussed relative to certain embodiments and figures, all embodiments of the present disclosure can include one or more of the features discussed herein. Further, while one or more embodiments may be discussed as having certain advantageous features, one or more of such features may also be used with the various embodiments discussed herein. In similar fashion, while exemplary embodiments may be discussed below as device, system, or method embodiments, it is to be understood that such exemplary embodiments can be implemented in various devices, systems, and methods of the present disclosure.

### BRIEF DESCRIPTION OF FIGURES

The following detailed description of specific embodiments of the disclosure will be better understood when read in conjunction with the appended drawings. For the purpose of illustrating the disclosure, specific embodiments are shown in the drawings. It should be understood, however, that the disclosure is not limited to the precise arrangements and instrumentalities of the embodiments shown in the drawings.

FIG. 1A illustrates a microfluidic device for buffer exchange with an angled ridge, according to aspects of the present disclosure.

FIG. 1B shows a detailed view of forces acting on particles in the microfluidic device of FIG. 1A, according to an embodiment.

FIG. 1C illustrates fluid flow patterns in the microfluidic device of FIG. 1A, according to aspects of the present disclosure.

FIG. 2A illustrates a perspective view of a microfluidic channel showing dimensional parameters, according to an embodiment.

FIG. 2B illustrates a perspective view of a microfluidic channel showing dimensional parameters, according to an embodiment.

FIG. 2C illustrates a top view showing ridge parameters of the microfluidic device, according to aspects of the present disclosure.

FIG. 3A depicts a graph showing collected cells versus cell suspension flowrate for different ridge angles, according to an embodiment.

FIG. 3B depicts a graph showing percent of total outlet cells versus ridge angle, according to aspects of the present disclosure.

FIG. 3C depicts a bar graph showing relative absorbance measurements for different outlet samples, according to an embodiment.

FIG. 4A depicts a graph showing relative cell count percentages across different outlets and cell concentrations, according to aspects of the present disclosure.

FIG. 4B depicts a graph showing relative cell counts for two cell concentrations, according to an embodiment.

FIG. 4C depicts a bar graph comparing viability percentages between cell inlet and outlet samples, according to aspects of the present disclosure.

FIG. 5A depicts a graph showing relative cell counts across five outlets at different flowrates, according to an embodiment.

FIG. 5B depicts a graph showing relative cell count data for outlet samples, according to aspects of the present disclosure.

### DETAILED DESCRIPTION

Although preferred exemplary embodiments of the disclosure are explained in detail, it is to be understood that other exemplary embodiments are contemplated. Accordingly, it is not intended that the disclosure is limited in its scope to the details of construction and arrangement of components set forth in the following description or illustrated in the drawings. The disclosure is capable of other exemplary embodiments and of being practiced or carried out in various ways. Also, in describing the preferred exemplary embodiments, specific terminology will be resorted to for the sake of clarity.

To facilitate an understanding of the principles and features of the present disclosure, various illustrative embodiments are explained below. The components, steps, and materials described hereinafter as making up various elements of the embodiments disclosed herein are intended to be illustrative and not restrictive. Many suitable components, steps, and materials that would perform the same or similar functions as the components, steps, and materials described herein are intended to be embraced within the scope of the disclosure. Such other components, steps, and materials not described herein can include, but are not limited to, similar components or steps that are developed after development of the embodiments disclosed herein.

As used in the specification and the appended claims, the singular forms “a,” “an” and “the” include plural referents unless the context clearly dictates otherwise.

Also, in describing the preferred exemplary embodiments, terminology will be resorted to for the sake of clarity. It is intended that each term contemplates its broadest meaning as understood by those skilled in the art and includes all technical equivalents which operate in a similar manner to accomplish a similar purpose.

Ranges can be expressed herein as from “about” or “approximately” one particular value and/or to “about” or “approximately” another particular value. When such a range is expressed, another exemplary embodiment includes from the one particular value and/or to the other particular value.

Similarly, as used herein, “substantially free” of something, or “substantially pure”, and like characterizations, can include both being “at least substantially free” of something, or “at least substantially pure”, and being “completely free” of something, or “completely pure”.

By “comprising” or “containing” or “including” is meant that at least the named compound, member, particle, or method step is present in the composition or article or method, but does not exclude the presence of other compounds, materials, particles, method steps, even if the other such compounds, material, particles, method steps have the same function as what is named.

Mention of one or more method steps does not preclude the presence of additional method steps or intervening method steps between those steps expressly identified. Similarly, it is also to be understood that the mention of one or more components in a device or system does not preclude the presence of additional components or intervening components between those components expressly identified.

The materials described as making up the various members of the invention are intended to be illustrative and not restrictive. Many suitable materials that would perform the same or a similar function as the materials described herein are intended to be embraced within the scope of the invention. Such other materials not described herein can include, but are not limited to, for example, materials that are developed after the time of the development of the invention.

Reference will now be made in detail to exemplary embodiments of the disclosed technology, examples of which are illustrated in the accompanying drawings and disclosed herein. Wherever convenient, the same references numbers will be used throughout the drawings to refer to the same or like parts.

Buffer exchange represents a fundamental unit operation in particle processing, particularly for biological cells and other suspended particles. Traditional methods for buffer exchange typically rely on centrifugation, where particles are pelleted, supernatant is removed, and particles are resuspended in fresh medium. Such centrifugation-based approaches may present several limitations including manual labor requirements, workflow interruptions due to batch processing, potential particle loss, and possible negative impacts on cell viability and behavior.

Microfluidic approaches offer an alternative to conventional centrifugation methods by enabling continuous, automated processing of suspended particles. The present disclosure describes a microfluidic device that utilizes laminar flow principles to achieve buffer exchange of suspended particles. In laminar flow conditions, different fluid streams flow in parallel without turbulent mixing, allowing for precise control of fluid interfaces and particle trajectories.

The microfluidic device employs an angled ridge structure positioned within a flow channel to guide particles from one fluid stream to another. The device operates by introducing two different fluids into separate inlets of a microfluidic channel. A first fluid contains the suspended particles in an original buffer or medium, while a second fluid comprises the desired replacement buffer or medium. Under laminar flow conditions, these fluids flow side-by-side through the channel while maintaining distinct fluid boundaries.

The angled ridge serves as a deflection element that redirects particles from the first fluid stream into the second fluid stream. The ridge may be positioned at a shallow angle relative to the flow direction, creating a gradual transition path for particles. The ridge includes a gap that allows the original fluid to pass underneath, above, or otherwise around the ridge, while particles are deflected along the ridge surface due to their size being larger than the gap dimension.

This approach enables continuous buffer exchange without the need for centrifugation or other batch processing steps. Particles may be transferred from their original suspension medium into a fresh buffer while maintaining their suspended state throughout the process. The method may provide advantages including reduced processing time, automated operation, maintained particle viability, and the ability to integrate with other microfluidic processing steps.

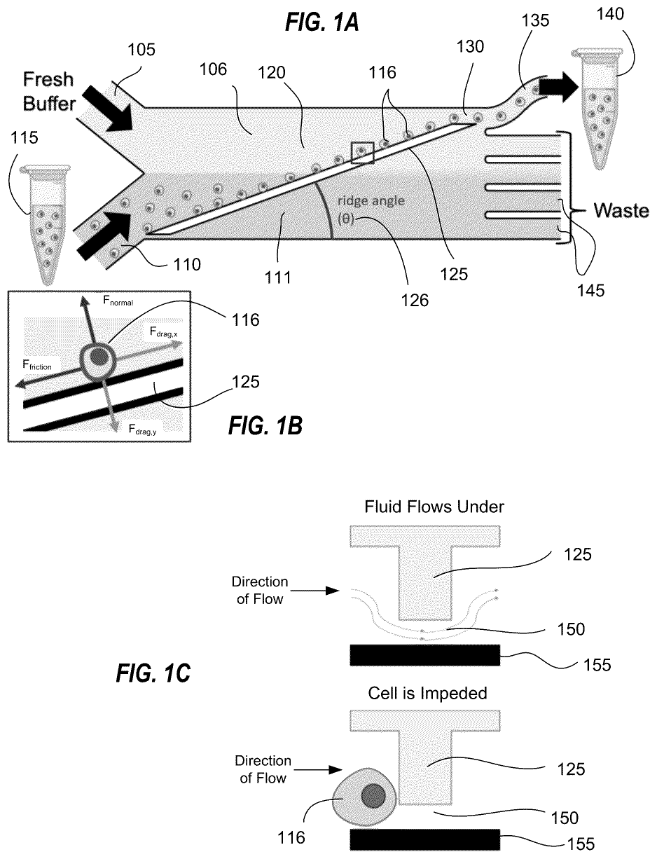

Referring to FIG. 1A-1C, a microfluidic device for buffer exchange of suspended particles may include several structural components arranged to facilitate continuous particle processing. The device may include a channel 120 that provides a flow path for fluids and particles through the microfluidic system. The channel 120 may have a first buffer inlet 105 configured to receive a first fluid containing suspended particles and a second buffer inlet 110 configured to receive a second fluid. A particle source 115 may be connected to or integrated with the first buffer inlet 105 to introduce particles 116 into the first buffer 106.

The channel 120 may contain an angled ridge 125 positioned within the channel at a ridge angle 126 relative to a channel wall 155. The ridge angle 126 may be 5 degrees or less relative to the channel wall 155 that extends parallel to a direction of flow of the first buffer 106 and second buffer 111. In some cases, the angled ridge 125 may extend partially across a width of the channel 120 and may be configured to deflect particles 116 from the first buffer 106 into the second buffer 111 while allowing the first buffer 106 and second buffer 111 to pass by the angled ridge 125 through a ridge gap 150.

As shown in FIG. 1A-1C, a gutter 130 may be positioned adjacent to a distal end of the angled ridge 125. The gutter 130 may be sized to allow the particles 116 to pass through the gutter region without obstruction. The ridge gap 150 may be formed between the angled ridge 125 and the channel wall 155, providing a passage for fluid flow while preventing particle passage due to the gap dimension being smaller than the particle diameter.

The microfluidic device may include multiple outlets for collecting different fluid streams and particles. A first buffer outlet 135 may be configured to collect the first buffer 106 after passage through the ridge gap 150. A particle collector 140 may serve as an outlet configured to collect particles 116 that have been transferred into the second buffer 111. Additionally, a waste outlet 145 may be provided to collect unwanted fluid portions or to facilitate flow control within the system.

The particles 116 that may be processed by the microfluidic device may include various types of suspended matter. In some cases, the particles 116 may comprise cells, such as biological cells used in research, diagnostic, or therapeutic applications. The particles 116 may also include droplets, solid beads, air bubbles, or other particles that may be suspended in flowable fluids. The particles can also be rigid or elastic and can have many different shapes, including, but not limited to, spherical, ellipsoidal, cubical, rod-like, and the like. The versatility of particle types allows the device to be applied across different fields including cell biology, materials processing, and analytical chemistry.

The first buffer 106 and second buffer 111 may comprise different fluid compositions depending on the specific buffer exchange application. The first buffer 106 may contain the original suspension medium, culture medium, or processing buffer in which the particles 116 are initially suspended. The second buffer 111 may comprise a replacement medium, fresh buffer solution, or alternative fluid composition into which the particles 116 are to be transferred.

With continued reference to FIG. 1A-1C, the microfluidic system for particle processing may include the microfluidic channel 120 having multiple inlets and multiple outlets as described above. An angled constriction element, which may correspond to the angled ridge 125, may be positioned within the channel 120 at an angle relative to fluid flow direction. The angled constriction element may have a gap dimension, corresponding to the ridge gap 150, that may be smaller than a diameter of particles 116 to be processed. A fluid delivery system may be configured to introduce two different fluids into the channel 120 through separate inlets, such as the first buffer inlet 105 and second buffer inlet 110. A collection system may be configured to collect processed particles from a designated outlet, such as the particle collector 140, where the angled constriction element may be configured to guide particles 116 from a first fluid stream into a second fluid stream while maintaining laminar flow separation between the fluid streams in the microfluidic channel 120.

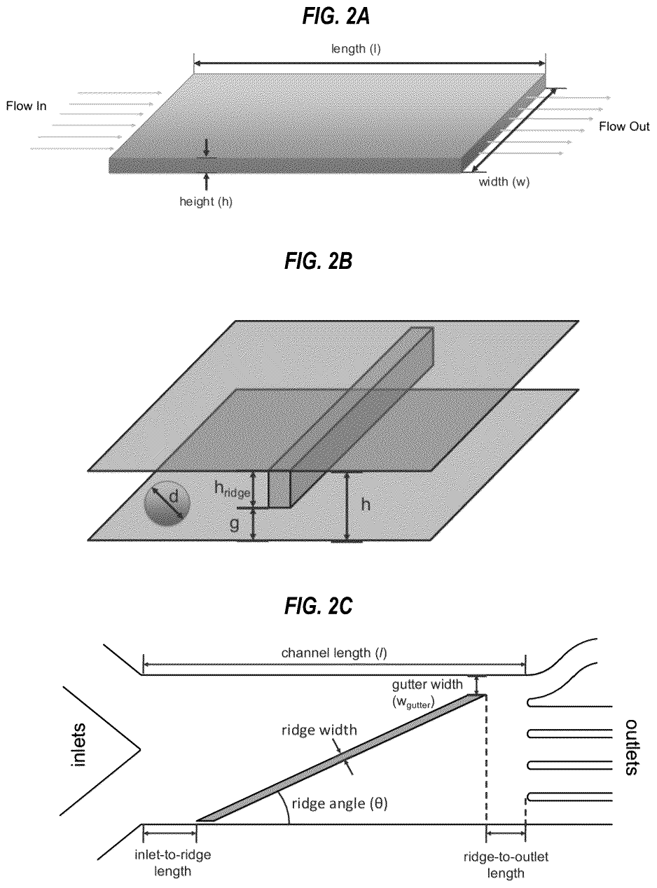

Referring to FIG. 2A-2C, the dimensional parameters of the microfluidic device may be configured to achieve effective buffer exchange while maintaining laminar flow conditions. FIG. 2A illustrates a perspective view of the microfluidic channel showing dimensional parameters including channel length (l), channel width (w), and channel height (h). FIG. 2B illustrates a perspective view showing dimensional parameters including channel height (h), ridge height (h_{ridge}), and gaps height (g). FIG. 2C illustrates a top view showing ridge parameters including gutter width (w_{gutter}), ridgth width, inlet-to-ridge length, ridge-to-outlet length, channel length (l), and ridge angle (θ) formed between the ridge and the horizontal plane.

The channel length—excluding the inlet-to-ridge and ridge-to-outlet lengths—may be determined by a trigonometric relationship that incorporates the channel width, gutter size, and ridge angle. This relationship may be expressed as

$$l = \frac{w - {w}_{gutter}}{tan ( θ )} ,$$

where l represents the channel length, w represents the channel width, w_{gutter} represents the gutter width, and θ represents the ridge angle. This mathematical relationship demonstrates how the geometric parameters interact to define the overall device dimensions.

As shown in FIG. 2B, the ridge angle θ may be 5 degrees or less relative to a channel wall parallel to the fluid flow direction. In some cases, the ridge angle may be 2 degrees or less or 1 degree or less relative to the channel wall. In some embodiments, however, the ridge angle may be greater than 5 degrees, such as 10 degrees, 15 degrees, 30 degrees, or larger. Larger angles can be utilized for separation of more rigid particles that have a lower propensity to deform through th gap. Lower ridge angles may reduce the fluid drag force component acting on particles in the direction perpendicular to the ridge. The perpendicular drag force component may act to deform particles through the ridge gap, and minimizing this force component may improve particle processing performance.

The ridge width can vary from 1 micron to 100 microns. In some embodiments, the ridgth width can be from 10 to 20 microns. The ridge height (h_{ridge}) can vary from 1 to 100 microns, typically from 5-50 microns, in accordance with various embodiments of the present disclosure. The inlet-to-ridge length can vary from 50-500 micons or larger, in accordance with various embodiments of the present disclosure. The channel length (l) can vary from 20 microns to 10 cm, or more typically from 50-500 microns, in accordance with various embodiments of the present disclosure.

The channel width w may range from 10 microns to 1 centimeter, in accordance with various embodiments of the present disclosure. The channel width may be minimized to minimize the channel length according to the trigonometric relationship described above. The channel height h may be larger than the largest particle size (e.g., 1 micron to 1 milimeter) to allow particle passage through the channel. In some embodiments, the channel height (h) can vary from 5 microns to 5 milimeters. The channel height may be minimized while maintaining this size constraint to maintain Reynolds numbers sufficiently low to maintain laminar flow conditions.

With continued reference to FIG. 2A and FIG. 2B, the gutter width w_{gutter} may be sized relative to the particles being processed. In some embodiments, the gutter width w_{gutter} can range from 1-100 microns or 10-50 microns. For cell processing applications, the gutter region may have a width that may be at least three times larger than an average diameter of the cells. This sizing relationship may prevent clogging of cells or particles within the gutter region. Larger gutter dimensions may reduce the chance of clogging from cells, particles, or foreign debris such as dust.

The ridge gap may have a width smaller than a smallest diameter of the suspended particles to prevent particles from passing underneath (or above or otherwise around) the angled ridge. The ridge gap width can vary from 1 micron to 1 milimeter or, more typically, 5-10 microns, in accordance with various embodiments of the present disclosure. For example, for cells having an average diameter of 15 micrometers, the ridge gap may be approximately 5 micrometers, which corresponds to approximately one-third of the average diameter of the suspended particles. This gap dimension may be effective for preventing cell passage while allowing fluid flow through the gap. The ridge gap dimension may account for particle deformability, where more deformable particles may require smaller gap sizes to prevent passage through the ridge gap.

A flow ratio constraint may govern the relationship between gutter width and total channel width to prevent crossover of the original buffer into the gutter region. This constraint may be expressed as w_{gutter}/w<Q_{fresh}/(Q_{fresh}+Q_{original}), where Q_{fresh} represents the flow rate of fresh buffer and Q_{original} represents the flow rate of original buffer. For successful buffer exchange, streamlines of the original buffer may not cross into the gutter, such that all flow through the gutter comprises fresh buffer. The ridge gap may not be too small as to prevent the original buffer from flowing through the ridge gap due to increased flow resistance.

The angled constriction element of the microfluidic system may be positioned at an angle of 5 degrees or less relative to a channel wall parallel to the fluid flow direction of the first and second fluid streams. In some cases, the angled constriction element may be positioned at an angle of 2 degrees or less or 1 degree or less relative to the channel wall. For cell processing applications, the gap dimension of the angled constriction element may be approximately one-third of an average diameter of the cells, providing effective particle deflection while maintaining fluid flow through the gap.

The method for continuous buffer exchange of suspended particles may operate through a series of coordinated fluid handling and particle manipulation steps. The method may begin by introducing a first fluid containing suspended particles into a first inlet of a microfluidic channel. The first fluid may comprise an original buffer, culture medium, or other suspension medium in which particles are initially dispersed. Simultaneously, a second fluid may be introduced into a second inlet of the microfluidic channel. The second fluid may comprise a replacement buffer, fresh medium, or alternative fluid composition into which particles are to be transferred.

Referring to FIGS. 1A-C, the first fluid and second fluid may flow in parallel within the channel under laminar flow conditions. Laminar flow conditions may be characterized by smooth, orderly fluid motion without turbulent mixing between adjacent fluid streams. Under these conditions, the first fluid and second fluid may maintain distinct boundaries while flowing side-by-side through the microfluidic channel. The Reynolds number of the flow may be maintained sufficiently low to preserve laminar flow characteristics throughout the buffer exchange process.

The suspended particles may be deflected from the first fluid into the second fluid using an angled ridge positioned within the channel. The angled ridge may be positioned at an angle of 5 degrees or less relative to a channel wall parallel to a direction of flow of the first and second fluids. In some cases, the angled ridge may be positioned at an angle of 4 degrees or less relative to the channel wall. In some cases, the angled ridge may be positioned at an angle of 3 degrees or less relative to the channel wall. In some cases, the angled ridge may be positioned at an angle of 2 degrees or less relative to the channel wall. In some cases, the angled ridge may be positioned at an angle of 1 degree or less relative to the channel wall. As discussed above, however, in some embodiments, the ridge angle can be greater than 5 degrees. The angled ridge may extend partially across a width of the channel, creating a deflection surface that guides particles from one fluid stream to another while allowing continued fluid flow.

As shown in FIG. 1B, forces acting on particles as the particles encounter the angled ridge may include drag forces from the flowing fluids and contact forces from the ridge surface. The shallow angle of the ridge may minimize the perpendicular force component acting on particles, reducing the tendency for particles to deform and pass through the ridge gap. The angled ridge may serve as a physical barrier that redirects particle trajectories from the first fluid stream into the second fluid stream.

The first fluid may pass by the angled ridge through a ridge gap while the particles are deflected along the angled ridge. As illustrated in FIG. 1C, the ridge gap may allow fluid flow underneath the ridge while preventing particle passage due to size exclusion. The ridge gap may be smaller than a smallest diameter of the suspended particles to prevent particles from passing through the ridge gap. For applications involving cells as the suspended particles, the ridge gap may have a width that may be approximately one-third of an average diameter of the cells. Though the ridge gap is positioned beneath the ride in FIG. 1C, the disclosure is not so limited. Rather, for example, in some embodiments, the ridge may extend upwards from a bottom surface of the channel, such that the ridge gap is located between a top surface of the ridge and a top surface of the channel 120.

The method may maintain the first fluid and second fluid under laminar flow conditions throughout the buffer exchange process. Flow rates may be controlled to maintain Reynolds numbers below the threshold for turbulent flow. The laminar flow regime may preserve the distinct boundaries between fluid streams and enable precise control of particle trajectories during the deflection process.

Particles suspended in the second fluid may be collected from an outlet of the channel following deflection by the angled ridge. The particles may be transferred from their original suspension medium into the second fluid while maintaining their suspended state. The collection outlet may be positioned to capture the second fluid stream containing the transferred particles, while separate outlets may collect the first fluid and any remaining fluid portions.

Channel surfaces may be coated with a surfactant to prevent cell adhesion during the buffer exchange process. The surfactant coating may reduce the tendency for cells or other particles to adhere to channel walls, ridge surfaces, or other internal surfaces of the microfluidic device. Such surface treatment may improve particle recovery rates and prevent clogging or fouling of the microfluidic channels during operation. The surfactant coating may be particularly beneficial when processing biological cells that may exhibit adhesive properties under certain conditions.

The method may enable continuous processing of suspended particles without the need for batch operations or centrifugation steps. Particles may be continuously introduced through the first inlet and continuously collected from the outlet, allowing for streamlined processing workflows. The buffer exchange process may maintain particle viability and functionality while achieving effective transfer from one fluid medium to another.

The microfluidic device may be configured to provide increased particle concentration functionality by collecting only a portion of the second fluid containing the deflected particles while discarding remaining portions of both the first fluid and second fluid. In this concentration mode, particles may be transferred from a distributed state throughout the first fluid into a concentrated state within a smaller volume of the second fluid. The method may further comprise a step of concentrating the particles by collecting only a portion of the second fluid containing the deflected particles while discarding remaining portions of both the first fluid and second fluid. This concentration approach may reduce the total fluid volume while maintaining or increasing the particle density in the collected output. Exemplary particle concentrations can range from 1 cell/mL to 10¹⁰ cells/mL, or more typically 10⁴ cell/mL to 10⁷ cells/mL.

The microfluidic device may function as a size filter by selectively allowing particles above a certain size threshold to be deflected by the angled ridge while permitting smaller particles to pass through the ridge gap. Particles larger than the gap dimension may be deflected along the ridge surface and transferred into the second fluid stream, while particles smaller than the gap dimension may pass through the ridge gap and remain in the first fluid stream. This size-based separation capability may enable simultaneous buffer exchange and particle fractionation based on size differences.

Multiple microfluidic devices may be arranged sequentially to create a processing chain for handling different particle sizes or transferring particles to different fluid compositions. A first device in the sequence may process particles of a first size range, while a second device may process particles of a different size range. In some embodiments, a device can have multiple ridges with differing lengths, such that each respective ridge ends at a different lateral location in the channel resulting in spatial separation of particles deflcted by different ridges. In some embodiments, the ridge length may increase with increasing distance from the inlet. Alternatively, sequential devices may transfer particles through a series of different buffer compositions, enabling multi-step buffer exchange processes. The sequential arrangement may provide enhanced processing capabilities beyond what may be achieved with a single device. For example, a third inlet can be utilized to introduce a third buffer fluid between the first and second buffers. In such a scenario, cells could exchange through multiple buffers as they travel along the ridge.

While the ridges shown in FIGS. 1-2C show ridges having a generally straight linear shape, the disclosure is not so limited. Rather, ridges can be curved, arc shaped, zig-zag shaped, composed or two or more straight segments, to enhance deflection of particles as they move along ridges. In some embodiments, ridges may have a chevron shape to focus particles toward opposing channel sidewalls.

The microfluidic system may be configured to process three fluids simultaneously, directing particles to different fluid streams based on particle size characteristics. A three-fluid configuration may include a first fluid containing mixed particle populations, a second fluid serving as a first collection medium, and a third fluid serving as a second collection medium. Particles above a first size threshold may be directed to the second fluid, while particles above a second size threshold may be directed to the third fluid. This multi-fluid approach may enable simultaneous size-based sorting and buffer exchange operations.

The microfluidic device may include multiple ridges positioned at different angles in different regions of the channel to enhance particle processing performance. A first ridge may be positioned at a first angle to deflect particles of a first size range, while a second ridge may be positioned at a second angle to deflect particles of a different size range. The multiple ridge configuration may provide staged particle processing, where particles encounter successive deflection elements with different geometric characteristics. Each ridge may have a different gap dimension and angle to optimize processing for specific particle populations. In such embodiments, different ridges can be utiliaed to direct particular particles to different outlets. The distance between consecutive ridges can vary, typically from 50-500 microns, though longer distances, e.g., up to 5 milimeters and more, can also be used.

The outlet resistances of the microfluidic device may be designed to be similar across different outlets to maintain consistent flow rates throughout the system. Balanced outlet resistances may prevent preferential flow through low-resistance pathways and may ensure uniform flow distribution among multiple outlet channels. The resistance balancing may be achieved through geometric design of outlet channels, including channel length, width, and cross-sectional area. Consistent flow rates across outlets may improve the predictability and reproducibility of particle processing results.

The fluid delivery system may comprise syringe pumps configured to maintain laminar flow conditions of the first and second fluid streams. Syringe pumps may provide precise flow rate control and may generate steady, pulsation-free fluid flow suitable for maintaining laminar flow regimes. The syringe pumps may be programmed to deliver specific flow rates for each fluid inlet, allowing for controlled flow rate ratios between the first fluid and second fluid. The controlled flow delivery may maintain Reynolds numbers below the threshold for turbulent flow, preserving the laminar flow conditions throughout the buffer exchange process.

Alternative fluid delivery systems may include peristaltic pumps, pressure-driven flow systems, or gravity-fed flow systems, depending on the specific application requirements. The fluid delivery system may include flow sensors or flow meters to monitor and adjust flow rates during operation. Feedback control systems may be integrated with the fluid delivery system to maintain target flow rates and compensate for variations in fluid properties or system resistance.

The microfluidic system may be integrated with automated sample handling equipment to enable high-throughput particle processing. Automated systems may include robotic sample loading, programmable fluid delivery sequences, and automated collection of processed samples. The integration may enable continuous operation with minimal manual intervention, supporting applications requiring processing of large sample volumes or multiple sample types.

Experimental validation of the microfluidic buffer exchange device demonstrates the relationship between ridge angle and device performance. Testing was conducted using cells suspended in PBS with blue food dye added as a model contaminant to evaluate both cell transfer efficiency and buffer purity during the exchange process.

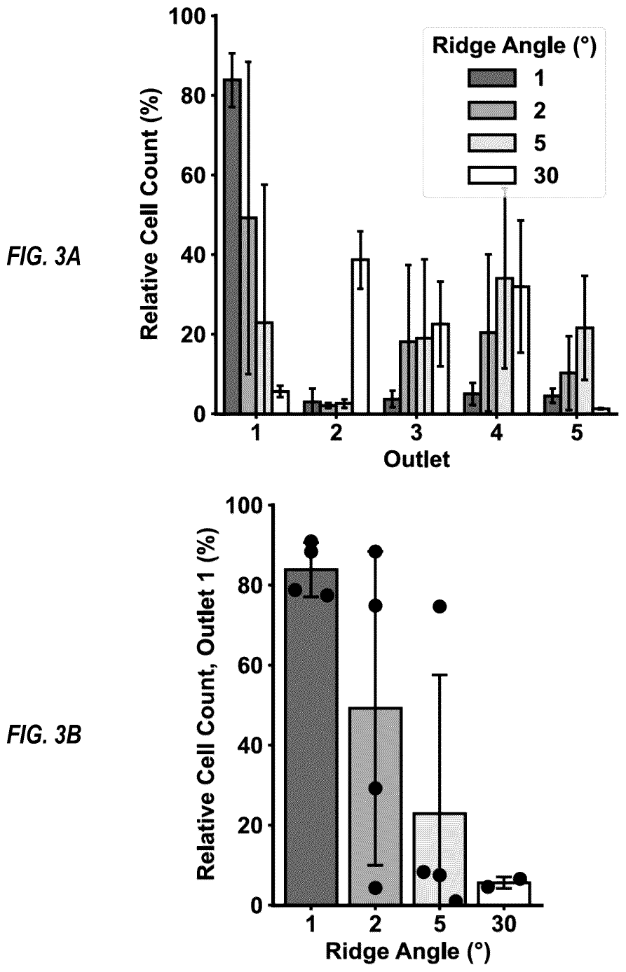

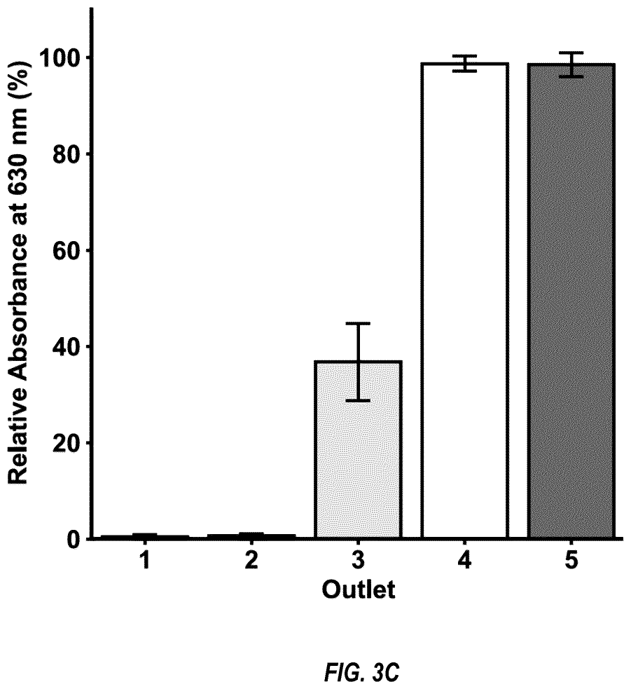

Referring to FIG. 3A and FIG. 3B, experimental results show the effect of ridge angle on cell collection performance across different operating conditions. FIG. 3A illustrates collected cells in each of 5 outlets as a percentage of the combined cell count from each outlet for ridge angles of 1, 2, 5, and 30 degrees. The data demonstrates that lower ridge angles provide improved cell collection efficiency at the tested flowrate of 20 μL/min through each of the two inlets (40 μL/min total). At ridge angles of 1 and 2 degrees, cell collection percentages remain relatively stable, while higher ridge angles of 5 and 30 degrees show decreased collection in outlet 1 (the target outlet) and greater variability across all outlets.

FIG. 3B shows the percent of total outlet cells collected in outlet 1 (the target outlet) versus ridge angle, providing a direct comparison of collection efficiency across the tested angle range. The results demonstrate a clear trend where cell collection efficiency increases as ridge angle decreases. Ridge angles of 1 and 2 degrees achieve substantially higher cell collection percentages compared to ridge angles of 5 and 30 degrees. The 2-degree ridge angle configuration achieves approximately 74.9% cell transfer into the target outlet, representing a significant improvement over higher angle configurations. In some embodiments, the 2-degree ridge angle configuration can achieve approximately 49.2% cell transfer into the target outlet, and the 5-degree ridge angle configuration can achieve approximately 22.8% cell transfer.

The improved performance at lower ridge angles may be attributed to reduced clogging and enhanced particle deflection characteristics. Higher ridge angles may create steeper deflection paths that increase the perpendicular force component acting on cells, potentially leading to cell deformation and passage through the ridge gap or accumulation at the ridge surface. Lower ridge angles may provide gentler deflection paths that guide cells along the ridge surface without excessive deformation or clogging.

With continued reference to FIG. 3A and FIG. 3B, the experimental data supports the selection of ridge angles of 5 degrees or less for effective buffer exchange operations. Ridge angles of 2 degrees or less may provide enhanced performance characteristics, including reduced clogging susceptibility and improved cell transfer efficiency.

Referring to FIG. 3C, buffer purity measurements demonstrate the effectiveness of the buffer exchange process in maintaining separation between the original cell suspension and fresh buffer. The bar graph shows relative absorbance measurements at 630 nm for different outlet samples from a cell suspension experiment conducted at a cell suspension flowrate of 20 μL/min. The absorbance measurements serve as indicators of contamination from the original buffer containing blue food dye.

The results in FIG. 3C show minimal absorbance values for outlet 1 and outlet 2, indicating low levels of contamination from the original cell suspension. Outlet 3 shows an intermediate absorbance value around 0.3, while outlet 4 and outlet 5 display higher absorbance values approaching 1.0. This distribution pattern demonstrates that fresh buffer transfer maintains minimal contamination from the original cell suspension, with the highest purity achieved in the outlets positioned furthest from the original buffer stream.

The buffer purity results confirm that the laminar flow conditions and ridge deflection mechanism effectively maintain separation between the original buffer and fresh buffer streams. The low contamination levels in the target collection outlets indicate that particles may be successfully transferred into fresh buffer while maintaining the chemical composition and purity of the replacement medium. This separation capability may be particularly important for applications requiring complete removal of original buffer components or contaminants.

The experimental validation demonstrates that the microfluidic buffer exchange device achieves both effective particle transfer and buffer purity maintenance through proper selection of ridge angle and operating conditions. The combination of reduced ridge angles and controlled flow conditions enables high-efficiency buffer exchange with minimal cross-contamination between fluid streams.

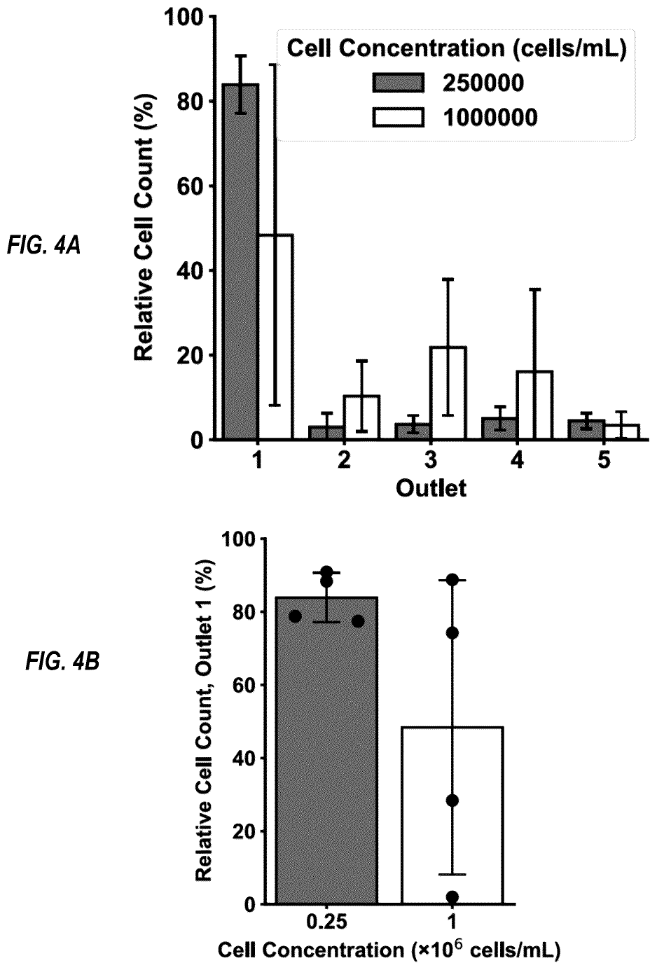

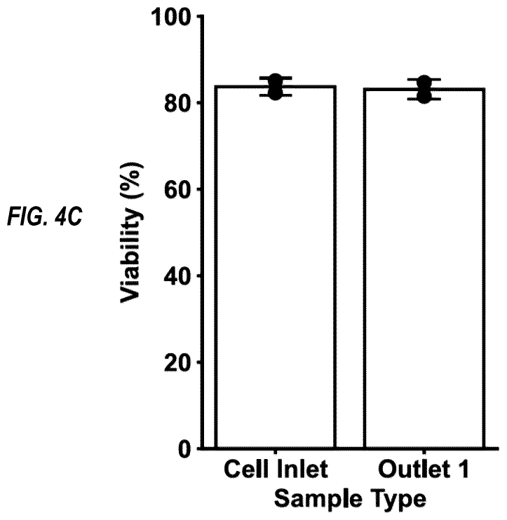

Referring to FIG. 4A and FIG. 4B, experimental results demonstrate the effect of cell concentration on device performance and particle distribution across multiple outlets. The microfluidic buffer exchange device was tested using two different cell concentrations to evaluate how input particle density affects processing efficiency and outlet distribution patterns.

FIG. 4A shows relative cell count percentages across outlets 1-5 for two different cell concentrations: 250,000 cells/mL and 1,000,000 cells/mL. The results demonstrate that the device maintains consistent performance characteristics across different input cell concentrations. Both concentration conditions show similar distribution patterns, with the majority of cells being collected in outlet 1, which corresponds to the target collection outlet for transferred particles. The higher concentration condition of 1,000,000 cells/mL shows slightly decreased collection efficiency in outlet 1 compared to the lower concentration of 250,000 cells/mL.

The distribution patterns shown in FIG. 4A indicate that the angled ridge deflection mechanism functions effectively across different particle densities. Outlets 2 through 5 show progressively lower cell counts, reflecting the successful deflection of particles from the original buffer stream into the fresh buffer stream. The consistency of distribution patterns between different cell concentrations suggests that the device may operate reliably across a range of input particle densities without requiring adjustment of operating parameters.

FIG. 4B presents relative cell count data for the same concentration conditions using alternative notation, showing results for 0.25×10\hat{ }6 cells/mL and 1×10\hat{ }6 cells/mL. The data confirms the trends observed in FIG. 4A, with both concentration conditions achieving effective particle transfer to the target outlet. The error bars shown in both graphs indicate measurement uncertainty and demonstrate the reproducibility of results across multiple experimental runs.

The concentration independence demonstrated in FIG. 4A and FIG. 4B may be attributed to the size-based deflection mechanism employed by the angled ridge. Since particle deflection depends primarily on particle size relative to the ridge gap dimension rather than particle concentration, the device may maintain consistent performance across different input densities. This concentration independence may provide operational flexibility for processing samples with varying particle densities without requiring device reconfiguration or parameter adjustment.

With continued reference to FIG. 4A and FIG. 4B, the experimental results support the scalability of the buffer exchange process for applications requiring processing of samples with different particle concentrations. The maintained distribution patterns across concentration conditions indicate that the laminar flow regime and particle deflection mechanisms remain stable regardless of input particle density. This stability may enable the device to handle samples with unknown or variable particle concentrations while maintaining predictable processing outcomes.

Referring to FIG. 4C, cell viability measurements demonstrate that the buffer exchange process maintains particle integrity throughout the processing operation. The bar graph compares viability percentages between the cell inlet and outlet 1 samples, showing over 80% viability for both sample types. The similar viability percentages between input and output samples indicate that the microfluidic buffer exchange process does not significantly impact cell health or functionality.

The viability results shown in FIG. 4C represent a significant advantage over traditional centrifugation-based buffer exchange methods, which may cause cell damage through mechanical stress, prolonged processing times, or exposure to centrifugal forces. The gentle deflection mechanism employed by the angled ridge may minimize mechanical stress on particles during the transfer process, preserving particle integrity and biological activity.

The maintained viability demonstrated in FIG. 4C may be attributed to several factors including the gentle deflection forces generated by the shallow ridge angle, the continuous flow processing that minimizes particle residence time, and the laminar flow conditions that avoid turbulent mixing or shear stress. The surfactant coating applied to channel surfaces may also contribute to maintained viability by preventing particle adhesion and associated damage during processing.

The error bars shown in FIG. 4C indicate the statistical variation in viability measurements and demonstrate the reproducibility of the viability preservation across multiple experimental runs. The consistent viability maintenance across different samples supports the reliability of the buffer exchange process for applications requiring preservation of particle functionality, such as cell culture, diagnostic assays, or therapeutic applications.

The combination of concentration-independent performance and maintained particle viability demonstrates that the microfluidic buffer exchange device may provide a robust alternative to conventional processing methods. The device may process samples across a range of particle concentrations while preserving particle integrity, enabling applications in research, diagnostics, and manufacturing where both processing efficiency and particle quality are important considerations.

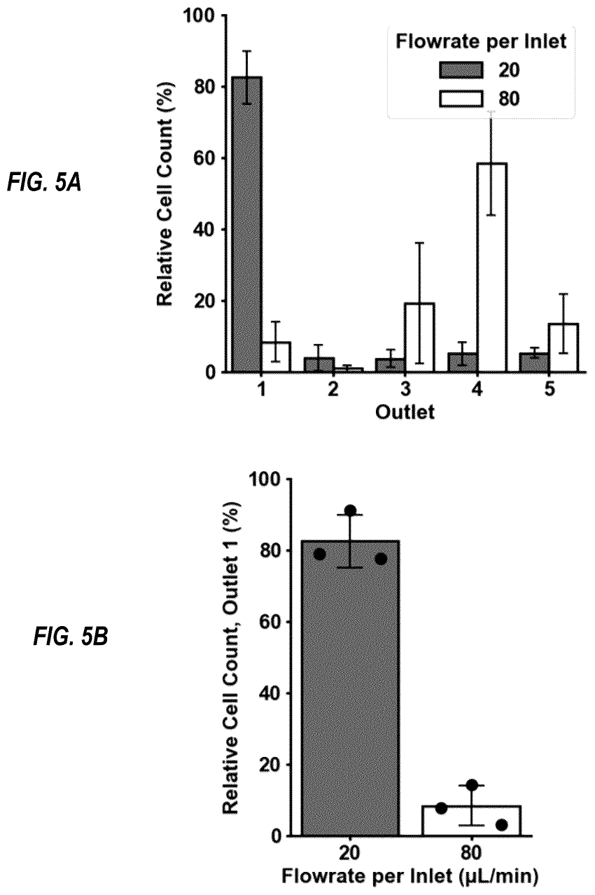

Referring to FIGS. 5A-5B, experimental results demonstrate the effect of flow rate variations on device performance and particle distribution characteristics. The microfluidic buffer exchange device was tested at two different flow rates per inlet to evaluate operational flexibility and processing robustness across different flow conditions.

FIG. 5A shows relative cell count data across five outlets at two different flowrates per inlet, with a dark gray bar representing a flowrate of 20 μL/min and a white bar representing a flowrate of 80 μL/min. The results demonstrate that the device maintains effective particle processing across both flow rate conditions. At the lower flow rate of 20 μL/min per inlet, the device achieves substantial cell collection in outlet 1, which corresponds to the target collection outlet for particles transferred into the fresh buffer stream. The higher flow rate condition of 80 μL/min per inlet shows a different distribution pattern, with reduced collection efficiency in outlet 1 but maintained overall particle processing capability.

The distribution patterns shown in FIG. 5A indicate that flow rate affects the efficiency of particle deflection and collection within the microfluidic system. The lower flow rate condition may provide more time for particles to interact with the angled ridge and undergo complete deflection into the target fluid stream. Higher flow rates may reduce the residence time available for particle deflection, potentially leading to incomplete transfer or altered distribution patterns across the outlet channels.

With continued reference to FIG. 5A, outlets 2 through 5 show varying cell counts depending on the flow rate condition. The 20 μL/min condition demonstrates a clear preference for collection in outlet 1, while the 80 μL/min condition shows more distributed collection across multiple outlets. This flow rate dependence may be attributed to changes in the fluid dynamics and particle trajectories within the channel as flow velocity increases.

FIG. 5B presents relative cell count data specifically for outlet 1, comparing the same two flowrate conditions of 20 and 80 μL/min per inlet. The data confirms that the lower flow rate of 20 μL/min achieves higher collection efficiency in the target outlet compared to the higher flow rate of 80 μL/min. The difference in collection efficiency between the two flow rate conditions demonstrates the importance of flow rate optimization for achieving maximum particle transfer efficiency.

The flow rate effects demonstrated in FIG. 5B may be related to the balance between particle deflection forces and fluid drag forces within the microfluidic channel. At lower flow rates, particles may have sufficient time to follow the deflection path created by the angled ridge and transfer completely into the target fluid stream. Higher flow rates may create stronger drag forces that compete with the deflection mechanism, potentially reducing the effectiveness of particle transfer.

The error bars shown in both FIG. 5A and FIG. 5B indicate measurement uncertainty and demonstrate the reproducibility of results across multiple experimental runs at each flow rate condition. The consistent error bar magnitudes across different flow rates suggest that the measurement precision remains stable regardless of the operating flow rate, supporting the reliability of the experimental data.

The operational flexibility demonstrated in FIGS. 5A-5B indicates that the microfluidic buffer exchange device may be operated across a range of flow rate conditions while maintaining particle processing capability. While collection efficiency may vary with flow rate, the device continues to function effectively at both tested conditions. This flow rate flexibility may enable adaptation of the device to different application requirements, such as high-throughput processing at elevated flow rates or high-efficiency processing at reduced flow rates.

The flow rate independence of basic device functionality, combined with the ability to optimize collection efficiency through flow rate selection, provides operational versatility for different processing scenarios. Applications requiring maximum particle recovery may benefit from lower flow rate operation, while applications prioritizing processing speed may accept reduced collection efficiency in exchange for higher throughput at elevated flow rates.

The experimental results support the scalability of the buffer exchange process across different flow rate regimes while maintaining the fundamental particle deflection and fluid separation mechanisms. The device may accommodate varying processing requirements through flow rate adjustment, providing a flexible platform for different buffer exchange applications.

The following examples pertain to further embodiments.

Embodiment 1 is a microfluidic device for buffer exchange of suspended particles, comprising a channel having a first inlet configured to receive a first fluid containing suspended particles and a second inlet configured to receive a second fluid; an angled ridge positioned within the channel at an angle of 5 degrees or less relative to a channel wall parallel to a direction of flow of the first and second fluids, the angled ridge extending partially across a width of the channel and configured to deflect particles from the first fluid into the second fluid while allowing the first and second fluids to pass by the angled ridge through a ridge gap; a gutter region adjacent to a distal end of the angled ridge, the gutter region sized to allow the particles to pass through the gutter region; and at least one outlet configured to collect particles transferred into the second fluid.

In Embodiment 2, the angled ridge of Embodiment 1 is positioned at an angle of 2 degrees or less relative to the channel wall.

In Embodiment 3, the suspended particles of Embodiment 1 comprise cells.

In Embodiment 4, the gutter region of Embodiment 3 has a width that is at least three times larger than an average diameter of the cells.

In Embodiment 5, the channel of Embodiment 1 has a width in a range of 100 microns to 1 millimeter.

In Embodiment 6, the ridge gap of Embodiment 1 has a width smaller than a smallest diameter of the suspended particles to prevent particles from passing underneath the angled ridge.

In Embodiment 7, the ridge gap of Embodiment 6 is approximately one-third of an average diameter of the suspended particles.

Embodiment 8 is a method for continuous buffer exchange of suspended particles, comprising introducing a first fluid containing suspended particles into a first inlet of a microfluidic channel; introducing a second fluid into a second inlet of the microfluidic channel, wherein the first fluid and second fluid flow in parallel within the channel under laminar flow conditions; deflecting the suspended particles from the first fluid into the second fluid using an angled ridge positioned within the channel at an angle of 5 degrees or less relative to a channel wall parallel to a direction of flow of the first and second fluids, wherein the angled ridge extends partially across a width of the channel; allowing the first fluid to pass by the angled ridge through a ridge gap while the particles are deflected along the angled ridge; and collecting the particles suspended in the second fluid from an outlet of the channel.

In Embodiment 9, the angled ridge of Embodiment 8 is positioned at an angle of 2 degrees or less relative to the channel wall.

In Embodiment 10, the suspended particles of Embodiment 8 comprise cells.

In Embodiment 11, the ridge gap of Embodiment 10 is smaller than a smallest diameter of the cells to prevent cells from passing through the ridge gap.

In Embodiment 12, the ridge gap of Embodiment 11 has a width that is approximately one-third of an average diameter of the cells.

In Embodiment 13, the first fluid and second fluid of Embodiment 8 are maintained under laminar flow conditions.

In Embodiment 14, the method of Embodiment 8 further comprises a step of concentrating the particles by collecting only a portion of the second fluid containing the deflected particles while discarding remaining portions of both the first fluid and second fluid.

Embodiment 15 is a microfluidic system for particle processing, comprising a microfluidic channel having multiple inlets and multiple outlets; an angled constriction element positioned within the channel at an angle relative to fluid flow direction, the angled constriction element having a gap dimension smaller than a diameter of particles to be processed; a fluid delivery system configured to introduce two different fluids into the channel through separate inlets; and a collection system configured to collect processed particles from a designated outlet, wherein the angled constriction element is configured to guide particles from a first fluid stream into a second fluid stream while maintaining laminar flow separation between the fluid streams in the microfluidic channel.

In Embodiment 16, the angled constriction element of Embodiment 15 is positioned at an angle of 5 degrees or less relative to a channel wall parallel to the fluid flow direction of the first and second fluid streams.

In Embodiment 17, the angled constriction element of Embodiment 16 is positioned at an angle of 2 degrees or less relative to the channel wall.

In Embodiment 18, the particles of Embodiment 15 comprise cells.

In Embodiment 19, the gap dimension of Embodiment 18 is approximately one-third of an average diameter of the cells.

In Embodiment 20, the fluid delivery system of Embodiment 15 comprises syringe pumps configured to maintain laminar flow conditions of the first and second fluid streams.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 wt. %” is intended to mean “about 40 wt. %”.

It is to be understood that the embodiments and claims disclosed herein are not limited in their application to the details of construction and arrangement of the components set forth in the description and illustrated in the drawings. Rather, the description and the drawings provide examples of the embodiments envisioned. The embodiments and claims disclosed herein are further capable of other embodiments and of being practiced and carried out in various ways. Also, it is to be understood that the phraseology and terminology employed herein are for the purposes of description and should not be regarded as limiting the claims.

Accordingly, those skilled in the art will appreciate that the conception upon which the application and claims are based may be readily utilized as a basis for the design of other structures, methods, and systems for carrying out the several purposes of the embodiments and claims presented in this application. It is important, therefore, that the claims be regarded as including such equivalent constructions.

Furthermore, the purpose of the foregoing Abstract is to enable the United States Patent and Trademark Office and the public generally, and especially including the practitioners in the art who are not familiar with patent and legal terms or phraseology, to determine quickly from a cursory inspection the nature and essence of the technical disclosure of the application. The Abstract is neither intended to define the claims of the application, nor is it intended to be limiting to the scope of the claims in any way.

1. A microfluidic device for buffer exchange of suspended particles, comprising:
a channel having a first inlet configured to receive a first fluid containing suspended particles and a second inlet configured to receive a second fluid;
an angled ridge positioned within the channel at an angle of 5 degrees or less relative to a channel wall parallel to a direction of flow of the first and second fluids, the angled ridge extending partially across a width of the channel and configured to deflect particles from the first fluid into the second fluid while allowing the first and second fluids to pass by the angled ridge through a ridge gap;
a gutter region adjacent to a distal end of the angled ridge, the gutter region sized to allow the particles to pass through the gutter region; and
at least one outlet configured to collect particles transferred into the second fluid.

2. The device of claim 1, wherein the angled ridge is positioned at an angle of 2 degrees or less relative to the channel wall.

3. The device of claim 1, wherein the suspended particles comprise cells.

4. The device of claim 3, wherein the gutter region has a width that is at least three times larger than an average diameter of the cells.

5. The device of claim 1, wherein the channel has a width in a range of 100 microns to millimeter.

6. The device of claim 1, wherein the ridge gap has a width smaller than a smallest diameter of the suspended particles to prevent particles from passing underneath the angled ridge.

7. The device of claim 6, wherein the ridge gap is approximately one-third of an average diameter of the suspended particles.

8. A method for continuous buffer exchange of suspended particles, comprising:
introducing a first fluid containing suspended particles into a first inlet of a microfluidic channel;
introducing a second fluid into a second inlet of the microfluidic channel, wherein the first fluid and second fluid flow in parallel within the channel under laminar flow conditions;
deflecting the suspended particles from the first fluid into the second fluid using an angled ridge positioned within the channel at an angle of 5 degrees or less relative to a channel wall parallel to a direction of flow of the first and second fluids, wherein the angled ridge extends partially across a width of the channel;
allowing the first fluid to pass by the angled ridge through a ridge gap while the particles are deflected along the angled ridge; and
collecting the particles suspended in the second fluid from an outlet of the channel.

9. The method of claim 8, wherein the angled ridge is positioned at an angle of 2 degrees or less relative to the channel wall.

10. The method of claim 8, wherein the suspended particles comprise cells.

11. The method of claim 10, wherein the ridge gap is smaller than a smallest diameter of the cells to prevent cells from passing through the ridge gap.

12. The method of claim 11, wherein the ridge gap has a width that is approximately one-third of an average diameter of the cells.

13. The method of claim 8, wherein the first fluid and second fluid are maintained under laminar flow conditions.

14. The method of claim 8, further comprising a step of concentrating the particles by collecting only a portion of the second fluid containing the deflected particles while discarding remaining portions of both the first fluid and second fluid.

15. A microfluidic system for particle processing, comprising:
a microfluidic channel having multiple inlets and multiple outlets;
an angled constriction element positioned within the channel at an angle relative to fluid flow direction, the angled constriction element having a gap dimension smaller than a diameter of particles to be processed;
a fluid delivery system configured to introduce two different fluids into the channel through separate inlets; and
a collection system configured to collect processed particles from a designated outlet, wherein the angled constriction element is configured to guide particles from a first fluid stream into a second fluid stream while maintaining laminar flow separation between the fluid streams in the microfluidic channel.

16. The system of claim 15, wherein the angled constriction element is positioned at an angle of 5 degrees or less relative to a channel wall parallel to the fluid flow direction of the first and second fluid streams.

17. The system of claim 16, wherein the angled constriction element is positioned at an angle of 2 degrees or less relative to the channel wall.

18. The system of claim 15, wherein the particles comprise cells.

19. The system of claim 18, wherein the gap dimension is approximately one-third of an average diameter of the cells.

20. The system of claim 15, wherein the fluid delivery system comprises syringe pumps configured to maintain laminar flow conditions of the first and second fluid streams.
